(19) United States
(12) Patent Application Publication
(10) Pub. No.: US 2014/0178427 A1
(43) Pub. Date: Jun. 26, 2014

(54) TOTAL POLYSACCHARIDES OF RADIX ISATIDIS AND THEIR FRACTIONS, AND USES THEREOF AS VACCINE ADJUVANTS

(71) Applicants: Junjie Shan, Beijing (CN); Yuxia Wang, Beijing (CN); Wei Jiang, Beijing (CN); Junhua Wu, Beijing (CN); Peiyuan Jia, Beijing (CN); Ting Zhu, Beijing (CN); Xiunan Zhao, Beijing (CN); Yulin Diao, Beijing (CN); Chenyu Wang, Beijing (CN)

(72) Inventors: Junjie Shan, Beijing (CN); Yuxia Wang, Beijing (CN); Wei Jiang, Beijing (CN); Junhua Wu, Beijing (CN); Peiyuan Jia, Beijing (CN); Ting Zhu, Beijing (CN); Xiunan Zhao, Beijing (CN); Yulin Diao, Beijing (CN); Chenyu Wang, Beijing (CN)

(21) Appl. No.: 13/883,486

(22) PCT Filed: Jun. 24, 2011
(86) PCT No.: PCT/CN2011/076310

§ 371 Date: Mar. 11, 2014

(30) Foreign Application Priority Data
Nov. 5, 2010 (CN) 201010533435.4
Jun. 22, 2011 (CN) 201110168725.8

(51) Int. Cl. A61K 39/39; A61K 47/36; A61K 39/145

(52) U.S. Cl. CPC A61K 39/39; A61K 39/145; A61K 47/36
USPC 424/209.1; 530/388.2; 530/389.1; 536/123.1; 514/777

(57) ABSTRACT

The present invention pertains to medicinal technical field, relates to a Radix isatidis total polysaccharide and fractions thereof and their uses as vaccine adjuvant. Specifically, it relates to a Radix isatidis total polysaccharide as well as neutral polysaccharide fraction and acidic polysaccharide fraction extracted from Chinese medicinal material Radix isatidis, and to their uses as vaccine adjuvant or uses in manufacture of vaccine composition. The present invention further relates to a vaccine adjuvant and vaccine preparation comprising the above Radix isatidis total polysaccharide or polysaccharide fraction, a method for preparing antibody, and a method for immunization or inoculation. The Radix isatidis total polysaccharide as well as neutral polysaccharide fraction and acidic polysaccharide fraction of the present invention all can be used as adjuvant for attenuated vaccines, protein vaccines, DNA vaccines or polypeptide vaccines.

### TECHNICAL FIELD

The present invention pertains to medical technical field, and relates to a Radix isatidis total polysaccharide and fractions thereof and their uses as vaccine adjuvant. Specifically, it relates to Radix isatidis total polysaccharide extracted from Chinese medicinal material, Radix isatidis, as well as neutral polysaccharides fraction and acidic polysaccharides fraction thereof, and to their uses as vaccine adjuvants or in manufacture of vaccine compositions. The present invention further relates to a vaccine adjuvant and vaccine preparation comprising the above Radix isatidis total polysaccharide or polysaccharides fraction, a method for preparing antibody, and a method for immunization or inoculation.

### BACKGROUND ART

Radix isatidis is dry root of Isatis indigotica of Cruciferae, has functions of clearing heat and toxic materials, cooling blood and relieving sore-throat, and is generally used for treatment of warm toxin with macula occurrence, dark red tongue with violet, sore throat, scarlatina, erysipelas and carbuncle. Modern pharmacological studies have shown that Radix isatidis can improve immune function and anti-tumor effect. Main chemical components of Radix isatidis comprise flavones, lignin, alkaloids and polysaccharides. In recent years, following documents report methods for preparing Radix isatidis total polysaccharide and their effects on animal immunologic functions.

QIU Yan, et al (Jiangsu Agriculture Science, 2009, 2: 32-35) reports a process for extracting Radix isatidis total polysaccharide (content of polysaccharides was 56%) by water decoction and alcohol precipitation, and a research about effects thereof on expression of mRNA of IL-4, IFN-γ in T lymphocytes of chicken peripheral blood. The results showed that the polysaccharides could elevate expression level of mRNA of IL-4, IFN-γ in lymphocytes. QIU Yan, et al (Journal of Nanjing Agricultural University, 2008, 31(1): 77-8) further report, when the polysaccharides were used in combination with Newcastle disease-infectious bronchitis combined low virulent strain (NDV-IBV) to immunize mice, Newcastle Disease HI antibody valence could be significantly improved, which could promote proliferation of peripheral blood T lymphocytes and elevate contents of CD4+, CD8+T lymphocytes and value of CD4+/CD8+.

KONG Xiangfeng, et al (Acta Veterinaria et Zootechnica Sinica, 2004, 35(4), 468-472) report Newcastle Disease IV series vaccines were used for immunizing chickings wherein high and low doses of Radix isatidis total polysaccharide (polysaccharide content was 82.94%) were injected before and after immunization, the results showed that antibody valence was elevated in different extents, and associated with administration time, dose and number of immunifaction.

ZHANG Hongying, et al (Journal of Henan Agricultural university, 2009, 43(2): 173-176) studied effects of Radix isatidis total polysaccharide in different concentrations on proliferation of in vitro cultured pig spleen lymphocytes. The results showed Radix isatidis total polysaccharide could significantly promote proliferation of pig spleen lymphocytes, could simultaneously synergize ConA or LPS to induce proliferation of pig spleen lymphocytes, and could significantly improve ConA induced secretion of IFN-γ from pig spleen lymphocytes, inhibit secretion of IL-2, and significantly promote secretion of NO.

Radix isatidis total polysaccharide as immunopotentiator in combination with an inactivated vaccine for porcine reproductive and respiratory syndrome were used to immunize piglets, and the results showed the polysaccharides could significantly elevate percentage of CD3+ and CD8+ lymphocytes and specific antibody titre of piglets (ZHANG Hongying, et al, Chinese Journal of Immunology, 2007, 23: 134-137).

Chen L, et al (Intervitrology, 2005, 48:207-212) disclosed Radix isatidis water decoction as adjuvant together with DNA vaccine of hand-foot-mouth disease virus (FMDV) was injected into mice, which could significantly improve antibody response of FMDV, promote proliferation of T cells, enhance protection ability of mice against hand-foot-mouth disease virus, and the effects thereof were superior to single injection of FMDV DNA.

CHEN Liang, et al (Chinese Patent, No.: ZL03145034.2, grant date: May 17, 2006) disclosed that when Radix isatidis water decoction as adjuvant was used in combination with foot-and-mouth disease virus DNA, hepatitis B virus core antigen prokaryotic expression product or foot-and-mouth disease inactivated vaccine, respectively, the specific antibody yield increased significantly. The adjuvant could indirectly or directly activate competent cells, increase antigen surface area, extend retention time of antigen on topic tissue.

LI Ning (Chinese Patent, publication No.: CN101703772A, publication date: May 12, 2010) disclosed manufacture of compound recipe of Radix isatidis oral liquid, in which Radix isatidis total polysaccharide was of 0.5-20 kg, astragalin crude drug was of 0.5-20 kg wherein astragalin was in an content of 50%, epimedium polysaccharide crude drug was in an amount of 0.5-10 kg wherein epimedium polysaccharide was in an content of 50%, morinda root extract was of 0.5-10 kg, potassium sorbate was of 50 g, and the residue was injection water. The oral liquid has good therapeutic effects for hypoimmunity caused by diseases or breeding conditions in birds, and could also enhance immune effects of vaccines.

Some documents also report methods for preparing Radix isatidis total polysaccharide.

CHEN Haoran, et al (Journal of China Dispensary, 2009, 20(21): 1642-1644) disclosed Radix isatidis was extracted with 8 times and 6 times water twice, aqueous extract was vacuum concentrated, added with ethanol to reach a final concentration of 70%, to obtain a precipitate as Radix isatidis crude polysaccharide fraction. The Radix isatidis total polysaccharide was dissolved in water, added with ethanol for fractional precipitation, to obtain 50% and 70% ethanol-precipitated polysaccharide fractions. The 70% ethanol-precipitated polysaccharide fraction was separated by Sephadex G100 exclusion chromatography to obtain purified Radix isatidis polysaccharide A. The Radix isatidis total polysaccharide A has a relative molecular weight of 11700, the polysaccharide A has two components after hydrolysis, i.e., arabinose and galactose.

ZHANG Tixiang, et al (Journal of Henan Institute of Engineering, 2009, 21(3): 13-17) used water-boiling alcohol-precipitating method to prepare crude Radix isatidis polysaccharide. The crude polysaccharide was then added with water for swelling, boiled and centrifuged, and the supernatant was added dropwise with an amount of trichloroacetic acid, stirred vigorously, centrifuged to remove precipitate, and subjected to dialysis, alcohol precipitation, washing and drying to obtain deproteinized polysaccharide. The polysaccharide was separated by dextran gel (Sephadex G-100) column chromatography to obtain an ISP2 polysaccharide with uniform molecular weight, whose relative molecular weight was 2.24×10⁵. ISP2 was a heteropolysaccharide consisting of four monosaccharides, i.e., rhamnose, fructose, glucose and galactose, and their mass ratio was 1:4:58.2:3.1.

ZHANG Tixiang, et al (Journal of Shizhen Chinese Medicine, 2009, 20 (8): 1992-1994) disclosed the following method for preparing refined polysaccharide: Radix isatidis→pulverization→weighing→degreasing with ethyl ether→extracting with hot water→centrifuging to obtain supernatant→extracting residue twice→combining supernatants→vacuum concentrating→dialyzing→measuring saccharide content→precipitating with ethanol→washing with organic solvent→vacuum drying→crude Radix isatidis polysaccharide. Crude polysaccharide solution→Sehadex G-100 column chromatography→concentrating eluent→precipitating with ethanol→lyophilizing→refined Radix isatidis total polysaccharide. The polysaccharide has a yield of 25.63%, and a polysaccharide content of 76.42%.

LU Jianjiang, et al (Journal of Guangdong Pharmacy, 2001, 11 (4): 16-18) disclosed Radix isatidis was elutriated, air dried, precisely weighed 100 g, placed in Soxhlet's extractor, extracted under refluxing in order with petroleum ether (60-90° C.), ethyl ether and 80% ethanol for 4 h. After the volatilization to remove solvent from residue, the residue was further extracted under refluxing with water for 4 h, vacuum concentrated to half volume, 0.1% activated carbon was added for decoloring, filtered. Then the filtrate was added with 95% ethanol so that the solution contained 80% of ethanol, and the solution was stood for overnight, filtered. The residue was washed with ethyl ether and anhydrous ethanol repeatedly, to obtain Radix isatidis total polysaccharide. The polysaccharide content as measured was 0.8099%.

However, the existing methods for preparing Radix isatidis total polysaccharide are relatively complex, have a relatively high cost, and may destroy active polysaccharide components thereof (especially in decocting and refluxing steps). In addition, the yield of Radix isatidis total polysaccharide is usually unsatisfied.

### CONTENTS OF THE INVENTION

The inventors did creative efforts and deep researches and obtained a Radix isatidis total polysaccharide and a polysaccharide fraction (neutral polysaccharide fraction and acidic polysaccharide fraction). In addition, the inventors surprisingly found the Radix isatidis total polysaccharide and the polysaccharide fraction could be used as good vaccine adjuvant. Hence, the following invention was provided.

One aspect of the present invention relates to a Radix isatidis polysaccharide fraction (i.e., neutral polysaccharide fraction) having the following characteristics:

(1) which comprises glucose, galactose, mannose, rhamnose, arabinose and xylose, and has molar ratio of Rha:Ara:Xyl:Man:Glc:Gal=1.00:2.35:2.38:9.27:27.47:13.03;

(2) expressed in glucose, saccharide content is 98.13%;

(3) molecular weight is 2000-10000.

Another aspect of the present invention relates to a Radix isatidis polysaccharide fraction (i.e., acidic polysaccharide fraction) having the following characteristics:

(1) which comprises arabinose, glucose, galactose, rhamnose and mannose, and has molar ratio of Rha:Ara:Man:Glc:Gal=1.00:40.06:0.61:22.24:18.04;

(2) expressed in glucose, saccharide content is 92.11%;

(3) expressed in galactose-uronic acid, glycuronic acid content is 6.41%;

(4) molecular weight is 3000-70000.

Further another aspect of the present invention relates to a Radix isatidis total polysaccharide, which comprises:

(1) the above Radix isatidis neutral polysaccharide fraction; and

(2) the above Radix isatidis acidic polysaccharide fraction.

The Radix isatidis total polysaccharide according to any one of items of the present invention is characterized in that:

(1) expressed in glucose, saccharide content is 58.93%;

(2) expressed in galactose-uronic acid, glycuronic acid content is 13.36%.

The Radix isatidis total polysaccharide according to any one of items of the present invention has characteristics as shown in FIG. 1 or FIG. 2.

The Radix isatidis total polysaccharide according to any one of items of the present invention is prepared by the following steps:

1) extracting Radix isatidis with water at 0° C.-60° C., to obtain a aqueous extract.

Preferably, extracting Radix isatidis with water was performed at 30° C.-60° C. or 25° C.-55° C.; more preferably, at 40° C.-55° C.; further preferably, at 45° C.-55° C.; particularly preferably, at 50° C.-55° C., for example, 50° C., 51° C., 52° C., 53° C., 54° C., or 55° C.

Without being limited with any theory, extracting temperature determines polysaccharide composition. If lower than 60° C., it generally does not influence chemical stability, but leads to low yield; if higher then 60° C., yield increases, but polysaccharide composition and polysaccharide structure may be influenced. In the range of 50° C.-55° C., it is well balanced in activity and yield for the prepared polysaccharide and polysaccharide fractions.

Extracting time is not specifically restricted, and preferably is 1-48 h, more preferably 2-12 h, further preferably 2-8 h, for example, 2, 3, 4, 5, 6, 7, or 8 h.

2) the aqueous extract of step 1) is subjected to ethanol precipitation, and the resulting supernatant is dialyzed and lyophilized, to obtain Radix isatidis total polysaccharide.

The Radix isatidis total polysaccharide according to any one of items of the present invention is characterized by any one or more of the following items (1)-(12):

(1) in step 1), the residue obtained after extraction is subjected to extraction under same conditions once or more times, and aqueous extracts are combined;

(2) in step 1), the used water is distilled water or deionized water;

(3) in step 1), the water is in an amount 5-15 times of Radix isatidis (L/Kg);

(4) in step 1), the used Radix isatidis is pulverized Radix isatidis;

(5) in step 1), the used Radix isatidis is Radix isatidis residue extracted with an organic solvent (such as, petroleum ether, ethyl acetate, chloroform, ethyl ether, n-hexane, cyclohexane, n-butanol, ethanol or methanol), (for example, extracted with 75% ethanol, and extraction may be performed for 24 h).

The portion of organic solvent extraction can be used for other purposes (e.g., for separation of other active small molecule components), which elevates the utilization rate of Radix isatidis raw material, and does not influence the extraction of polysaccharide and polysaccharide fractions.

(6) in step 1), stirring is performed during extraction period;

(7) in step 1), the obtained aqueous extract is subjected to vacuum concentration to obtain a concentrated aqueous extract;

(8) in step 2), conditions for ethanol precipitation are: after ethanol precipitation, final ethanol concentration is 60-80%; preferably, ethanol precipitation time is greater than 12 h;

(9) in step 2), the precipitate obtained by centrifugation after ethanol precipitation is further subjected to ethanol precipitation once or more times, and the resulting supernatants are combined;

(10) in step 2), dialysis bag used for dialysis has molecular cut off of greater than 1000;

the dialysis bag of the molecular weight range can effectively trap polysaccharides and oligosaccharides;

(11) in step 2), the dialysis is performed once or more times;

(12) in step 2), before lyophilization, the obtained dialysate is concentrated (for example, vacuum concentrated) at 50° C.-55° C.

The present invention further relates to a Radix isatidis total polysaccharide obtained by the above preparation method. In a specific embodiment, the Radix isatidis total polysaccharide has characteristics of the Radix isatidis total polysaccharide of any one of the above items.

The Radix isatidis neutral polysaccharide fraction according to any one of items of the present invention is prepared by the following steps:

subjecting the Radix isatidis total polysaccharide of any one of items of the present invention to DEAE-cellulose column chromatography to obtain a water elution portion.

The present invention further relates to the Radix isatidis neutral polysaccharide fraction as prepared according to the above preparation method. In a specific embodiment, the Radix isatidis neutral polysaccharide fraction has the characteristics of the Radix isatidis neutral polysaccharide fraction of any one of the above items.

The Radix isatidis polysaccharide fraction according to any one of items of the present invention is prepared by the following steps:

subjecting the Radix isatidis total polysaccharide of any one of items of the present invention to DEAE-cellulose column chromatography to obtain a 0.25 NaHCO₃ elution portion.

The present invention further relates to the Radix isatidis acidic polysaccharide fraction as prepared according to the above preparation method. In a specific embodiment, the Radix isatidis acidic polysaccharide fraction has the characteristics of the Radix isatidis acidic polysaccharide fraction of any one of the above items.

The further aspect of the present invention relates to a pharmaceutical composition, which comprises the Radix isatidis polysaccharide fraction or Radix isatidis total polysaccharide of any one of items of the present invention; optionally, further comprises a pharmaceutically acceptable excipient.

The further aspect of the present invention relates to an vaccine adjuvant, which comprises the Radix isatidis polysaccharide fraction or Radix isatidis total polysaccharide of any one of items of the present invention; specifically, the vaccine adjuvant is an adjuvant for attenuated vaccines, protein vaccines, DNA vaccines or polypeptide vaccines.

The further aspect of the present invention relates to a vaccine preparation or vaccine composition, which comprises the Radix isatidis total polysaccharide or Radix isatidis polysaccharide fraction.

The vaccine preparation or vaccine composition according to any one of items of the present invention is an attenuated vaccine, protein vaccine, DNA vaccine or polypeptide vaccine; specifically, is H1N1 influenza vaccine.

The further aspect of the present invention relates to a use of the Radix isatidis polysaccharide fraction or Radix isatidis total polysaccharide of any one of items of the present invention as a vaccine adjuvant; or a use in manufacture of a vaccine preparation, vaccine composition, or antibody.

The use according to any one of items of the present invention, in which the vaccine preparation is an attenuated vaccine, protein vaccine, DNA vaccine or polypeptide vaccine. The vaccine adjuvant is an adjuvant for attenuated vaccines, protein vaccines, DNA vaccines or polypeptide vaccines.

The further aspect of the present invention relates to a method for preparing an antibody, comprising a step of using an effective amount of the Radix isatidis polysaccharide fraction and/or Radix isatidis total polysaccharide of the present invention. Specifically, the antibody is a monoclonal antibody or polyclonal antibody.

The further aspect of the present invention relates to a method for immunization or vaccination, comprising administering a mammal with an effective amount of the vaccine preparation or vaccine composition of the present invention. In one embodiment of the present invention, the mammal is a human. Specifically, the vaccine preparation or vaccine composition is an attenuated vaccine, protein vaccine, DNA vaccine or polypeptide vaccine; more specifically, is H1N1 influenza vaccine. The amount should be determined by a doctor in a reliable medical judgment.

In the present invention, in absence of special explanation, the term “Radix isatidis polysaccharide fraction” refers to a Radix isatidis neutral polysaccharide fraction and/or acidic polysaccharide fraction.

The term “effective amount” refers to a dose of vaccine preparation or vaccine composition that can achieve immune effects.

### BENEFICIAL EFFECTS

Both the Radix isatidis total polysaccharide of the present invention and neutral polysaccharide fraction and acidic polysaccharide fraction thereof can be used as adjuvant for attenuated vaccine, protein vaccine, DNA vaccine or polypeptide vaccine. The Radix isatidis total polysaccharide or polysaccharide fractions of the present invention can be used as good vaccine adjuvant. In addition, the method for preparing the Radix isatidis total polysaccharide and polysaccharide fraction of the present invention is simple in process, has low cost and high yield, and thus facilitates production in large scale.

### BRIEF DESCRIPTION OF THE DRAWINGS

FIG. 1: DEAE-cellulose column chromatography elution curve of Radix isatidis total polysaccharide A (polysaccharide fraction was measured by phenol-sulfuric acid method, at wavelength of 490 nm).

FIG. 2: DEAE-cellulose column chromatography elution curve of Radix isatidis total polysaccharide A (optical absorbance as measured at wavelength of 280 nm).

FIG. 3: blood serum antibody titre of mouse after the 1^{st} immunization of OVA combined with BLG-A. mean±SD; n=5.

FIG. 4: blood serum antibody titre of mouse after the 2^{nd} immunization of OVA combined with BLG-A. mean±SD; n=5.

FIG. 5: blood serum antibody titre of mouse after the 3^{rd} immunization of OVA combined with BLG-A. mean±SD; n=5.

FIG. 6: blood serum antibody titre of mouse after the 1^{st} immunization of OVA combined with BLG-A (50-55° C.). mean±SD; n=5. (BLG-A: 1 mg/mouse; OVA 0.06 mg/mouse).

FIG. 7: blood serum antibody titre of mouse after the 2^{nd} immunization of OVA combined with BLG-A (50-55° C.). mean±SD; n=5. (BLG-A: 1 mg/mouse; OVA 0.06 mg/mouse).

FIG. 8: blood serum antibody titres of mouse after the 1^{st} immunization of OVA combined with BLG-A1, BLG-A2. mean±SD; n=5.

FIG. 9: blood serum antibody titres of mouse after the 2^{nd} immunization of OVA combined with BLG-A1, BLG-A2. mean±SD; n=5.

FIG. 10: blood serum antibody titres of mouse after the 3^{rd} immunization of OVA combined with BLG-A1, BLG-A2. mean±SD; n=5.

FIG. 11: blood serum antibody titre of mouse after the 1^{st} immunization of H1N1 combined with BLG-A1. H1N1:3 μg/mouse, BLG-A1: 1 mg/mouse. mean±SD; n=5.

FIG. 12: blood serum antibody titre of mouse after the 1^{st} immunization of H1N1 combined with BLG-A2. H1N1:3 μg/mouse, BLG-A2: 0.1 mg/mouse. mean±SD; n=5.

FIG. 13: antibody titre of mouse immunized with BLG-A1 and H1N1 attenuated vaccine (A1:10 mg/ml, 1 mg/mouse).

FIG. 14: antibody titre of mouse immunized with BLG-A2 and H1N1 attenuated vaccine (A2: 10 mg/ml, 1 mg/mouse).

### SPECIFIC MODELS FOR CARRYING OUT THE INVENTION

The embodiments of the present invention are illustrated in conjunction with the following examples, but those skilled in the art would understand the following examples are merely used to illustrate the present invention and should not be deemed to restrict the scope of the present invention. Those which are not specifically described in examples are performed according to conventional conditions or recommendations of manufacturers. The reagents or instruments of which manufacturers are not given are all commercially available conventional products.

### Example 1

### Preparation of Radix Isatidis Total Polysaccharide BLG-A Sample 1

Chinese medicinal material Radix isatidis 1 kg was pulverized, added at room temperature to 10 L of 75% ethanol and soaked for 24 h, filtered, centrifuged (3000 r/min×10 min), the residue was extracted under the same conditions once, the filtrates were combined, subjected to vacuum concentration at 40° C.-45° C. and the extractum was recovered. The Radix isatidis residue after extraction with 75% ethanol was dried at 50° C., added with 15 L of distilled water, extracted at room temperature for 24 h, stirred during the period; then filtered, the filtrate was centrifuged for 10 min (rotation rate: 3000 r/min), the Radix isatidis residue after extraction was subjected to the second extraction under the same conditions. The aqueous extracts of the two extractions were combined, vacuum concentrated at 50° C.-55° C. to reach 1000 ml, then added with 3 times volume (3000 ml) of 95% ethanol to perform alcohol precipitation for 48-72 h. The solution after alcohol precipitation was centrifuged (3000 r/min×10 min), the precipitate portion was added with 1000 ml of water and dissolved under stirring, centrifuged, the precipitate was subjected to the same operation twice. All supernatants of dissolution were combined, placed in dialysis bag (molecular cut off>1000), dialysed with tap water for 48 h, then with distilled water for 24 h. The dialysis solution was vacuum concentrated at 50° C.-55° C. to reach about 200 ml, placed in vials for lyophilization, to obtain a light yellow powder, i.e., Radix isatidis total polysaccharide BLG-A sample 1 (yield was 0.417%).

### Example 2

### Preparation of Radix Isatidis Total Polysaccharide BLG-A Sample 2

Chinese medicinal material Radix isatidis 1 kg was pulverized, added to 15 L of distilled water, extracted at 50° C.-55° C. for 4 h, stirred during the period; then filtered, the filtrate was centrifuged for 10 min (rotation rate: 3000 r/min). The Radix isatidis residue after extraction was subjected to the second extraction under the same conditions. The aqueous extracts of the two extractions were combined, vacuum concentrated at 50° C.-55° C. to reach 1000 ml, then added with 3 times volume (3000 ml) of 95% ethanol to perform alcohol precipitation for 48-72 h. The alcohol precipitation solution was centrifuged (3000 r/min×10 min), the precipitate portion was added with 1000 ml of water and dissolved under stirring, centrifuged, the precipitate was subjected to the same operation twice. All supernatants of dissolution were combined, placed in dialysis bag (molecular cut off>1000), dialysed with tap water for 48 h, then with distilled water for 24 h. The dialysis solution was vacuum concentrated at 50° C.-55° C. to reach about 200 ml, placed in vials for lyophilization to obtain a light yellow powder, i.e., Radix isatidis total polysaccharide BLG-A sample 2 (yield was 0.438%).

In comparison with the method for preparing sample 1, the method for preparing sample 2 is the same, except that the raw material for preparing sample 1 was the Radix isatidis residue after extraction with 75% ethanol, of which the objective is to obtain an extractum for other uses. However the inventors found in the following experiments that this did not influence the composition and effects of the prepared Radix isatidis total polysaccharide product.

### Example 3

### Preparation of Radix Isatidis Neutral Polysaccharide Fraction (BLG-A1) and Acidic Polysaccharide Fraction (BLG-A2)

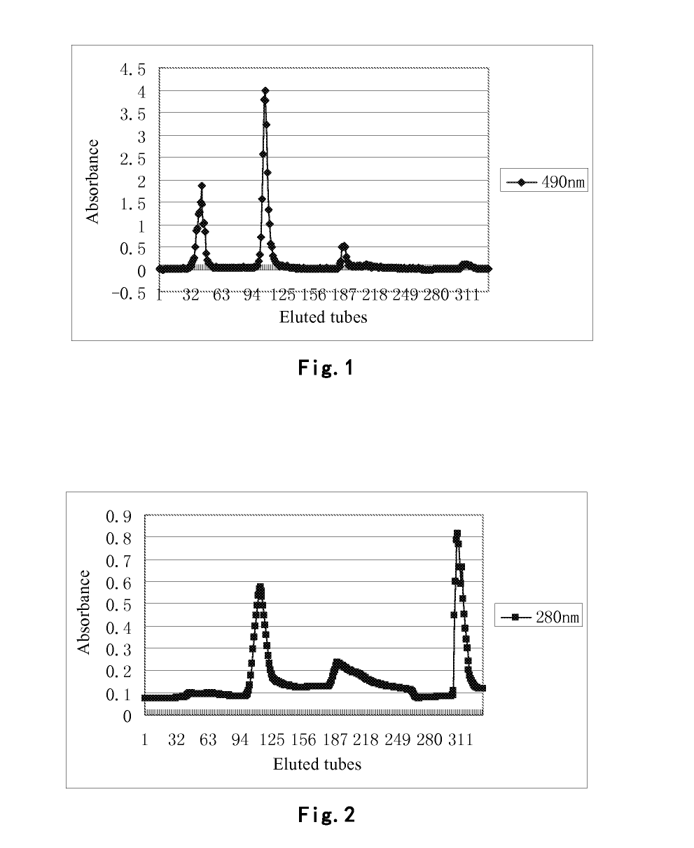

1 g of the Radix isatidis total polysaccharide as prepared in Example 2 was weighed, added to 50 ml of distilled water for dissolution. The dissolution solution was loaded to DEAE-cellulose column (Φ8 cm×35 cm), continuous elution was performed separately with water, 0.25 mol/L NaHCO₃, 0.5 mol/L NaHCO₃ and 0.1 mol/L NaOH, and polysaccharide fraction was measured by sulfuric acid-phenol method (FIG. 1), to correspondingly obtain polysaccharide fractions BLG-A1 (H₂O), BLG-A2 (0.25 mol/L NaHCO₃), BLG-A3 (0.5 mol/L NaHCO₃) and BLG-A4 (0.1 mol/L NaOH), and optical absorbencies at 280 nm of eluents were also measured at the same time, the elution curve was shown in FIG. 2.

### Example 4

### Determination of Physicochemical Properties of Radix Isatidis Total Polysaccharide, Neutral Polysaccharide Fraction and Acidic Polysaccharide Fraction

Experimental Samples:

The used Radix isatidis total polysaccharide was prepared in Example 2, the neutral polysaccharide fraction and acidic polysaccharide fraction were prepared in Example 3.

1. Determination of Saccharide Contents (Expressed in Glucose) of Radix Isatidis Total Polysaccharide, Neutral Polysaccharide Fraction and Acidic Polysaccharide fraction

1) Experimental methods

Saccharide content was determined by sulfuric-phenol method.

2) Experimental Results

The Radix isatidis total polysaccharide BLG-A was a light yellow powder and had saccharide content of 58.93% (expressed in glucose).

The neutral polysaccharide fraction BLG-A1 was a white powder and had saccharide content of 98.13% (expressed in glucose).

The acidic polysaccharide fraction BLG-A2 was a light yellow powder and had saccharide content of 92.11% (expressed in glucose).

2. Determination of Glycuronic Acid Contents (Expressed in Galactose-Uronic Acid) of Radix Isatidis Total Polysaccharide, Acidic Polysaccharide Fraction

1) Experimental Methods

Glycuronic acid content was determined by m-hydroxyl-biphenyl method.

2) Experimental Results

The glycuronic acid content of the Radix isatidis total polysaccharide BLG-A was 13.36% (expressed in galactose-uronic acid).

The glycuronic acid content of the acidic polysaccharide fraction BLG-A2 was 6.41% (expressed in galactose-uronic acid).

3. Determination of Monosaccharide Ratios of Radix Isatidis Neutral Polysaccharide Fraction and Acidic Polysaccharide Fraction

1) Experimental Methods

The monosaccharide ratios were obtained by derivation and gas chromatographic analysis.

2) Experimental Results

The neutral polysaccharide fraction BLG-A1 was mainly composed of glucose, galactose, mannose and small amounts of rhamnose, arabinose and xylose, in which the molar ratio of Rha:Ara:Xyl:Man:Glc:Gal=1.00:2.35:2.38:9.27:27.47:13.03.

The acidic polysaccharide fraction BLG-A2 was mainly composed of arabinose, glucose, galactose and small amounts of rhamnose and mannose, in which the molar ratio of Rha:Ara:Man:Glc:Gal=1.00:40.06:0.61:22.24:18.04.

4. Measurement of Molecular Weight of Radix Isatidis Neutral Polysaccharide Fraction and Acidic Polysaccharide Fraction

1) Experimental Methods

Instruments: HPLC, Waters Company; chromatography column: TSKsw 4000; mobile phase: 0.1 M Na₂SO4; flow rate: 0.6 ml/min; detector: differential detector.

2) Experimental Results

Molecular weight of neutral polysaccharide fraction BLG-A1 was 2000-10000.

Molecular weight of acidic polysaccharide fraction BLG-A2 was 3000-70000.

### Example 5

### Measurement of Adjuvant Activity of Radix Isatidis Total Polysaccharide Sample 1

1. Experimental Objective:

The Radix isatidis total polysaccharide BLG-A as prepared in Example 1 was used as adjuvant, ovalbumin (OVA) was used as antigen, both of them in combination were intramuscularly injected to mice, to measure the generated antibody titre.

2. Experimental Methods

Experimental animals: Balb/C, 6-8 weeks, 5 mice per group, female.

Drug concentration: the Radix isatidis total polysaccharide BLG-A as prepared in Example 1: 20 mg/ml; OVA: 1.2 mg/ml; aluminum adjuvant: 2 mg/ml;

Control solvent: physiological saline

Administration dose: OVA-60 μg/50 μl/mouse; aluminum adjuvant-100 μg/50 μl/mouse;

BLG-A: 1 mg/50 μl/mouse;

Grouping: (1) P group: PBS+OVA; (2) aluminum adjuvant group: aluminum+OVA; (3) BLG-A group: BLG-A+OVA; (4) solvent control group: physiological saline.

Mixing in equal-volume was performed before injection, 100 μl/mouse, intramuscular injection was performed at right hindlimb.

Immunization schedule: 3 weeks after animals were grouped for immunization and subjected to first immune injection, blood samples were taken from caudal vein, and antibody titre in blood serum was measured. Antibody titre was measured in the 3^{rd} week after first immune injection, booster immunization was performed in the 4^{th} week, 2 weeks after the second immune injections, blood samples were taken from caudal vein, antibody titre in blood serum was measured, and according to titre, the 3^{rd} immunization was performed after 2 weeks of measurement. Antibody titre was measured by ELISA method.

Preparation of reagents for ELISA method:

Antibody coating solution: 50 mmol/L carbonate buffer solution with pH of 9.6. Anhydrous Na₂CO₃ 1.696 g, NaHCO₃ 2.856 g were weighed, added with 1000 ml of water for dissolution, and pH was regulated to 9.6.

Cleaning solution (10×PBST, pH7.4): NaCl 80 g, KCl 2 g, Na₂HPO₄ 29 g, KH₂PO₄ 2 g, Tween-20 10 ml were weighed or taken, added with double distilled water to reach 1000 ml, regulated to have pH of 7.4, and diluted 10 time for use.

Confining liquid: 1% BSA, dissolved with 50 mmol/L PBS pH 7.4.

Substrate solution (TMB-H₂O₂): substrate solutions A and B were mixed in equal-volume for use, added with 30% H₂O₂ to have a final concentration of 0.5%.

Substrate solution A (TMB), weighed TMB 200 mg, dissolved in anhydrous ethanol 100 ml, added with double-distilled water to reach 1000 ml.

Substrate solution B (0.1 mol/L citric acid-0.2 mol/L Na₂HPO₄ buffer solution), Na₂HPO₄ 24.8 g, citric acid 19.33 g, added with double-distilled water to reach 1000 ml, regulated to have pH of 5.0-5.4.

2 N H₂SO₄

OVA was dissolved in antigen coating solution, and the concentration of it was 4 μg/ml. Coating was performed on 96-well plate (Costa) at 100 μl/well, 4° C. overnight. The 96-well plate was washed with PBST for 3 times, confined with 1% BSA-PBS at 37° C. for 1 h. After being washed with PBST for 3 times, the 96-well plate was added with mouse blood serum sample at 100 μl/well that was diluted with PBST, incubated at 37° C. for 1 h. Washed with PBST for 3 times, added with 100 μl/well of HRP-goat anti-mouse IgG (1:1000, PBST), incubated at 37° C. for 1 h, and washed with PBST for 6 times, added with 100 μl/well of substrate solution for coloration, then added with 50 μl/well of 2 N H₂SO₄ to terminate reaction and determine A₄₅₀.

3. Experimental Results

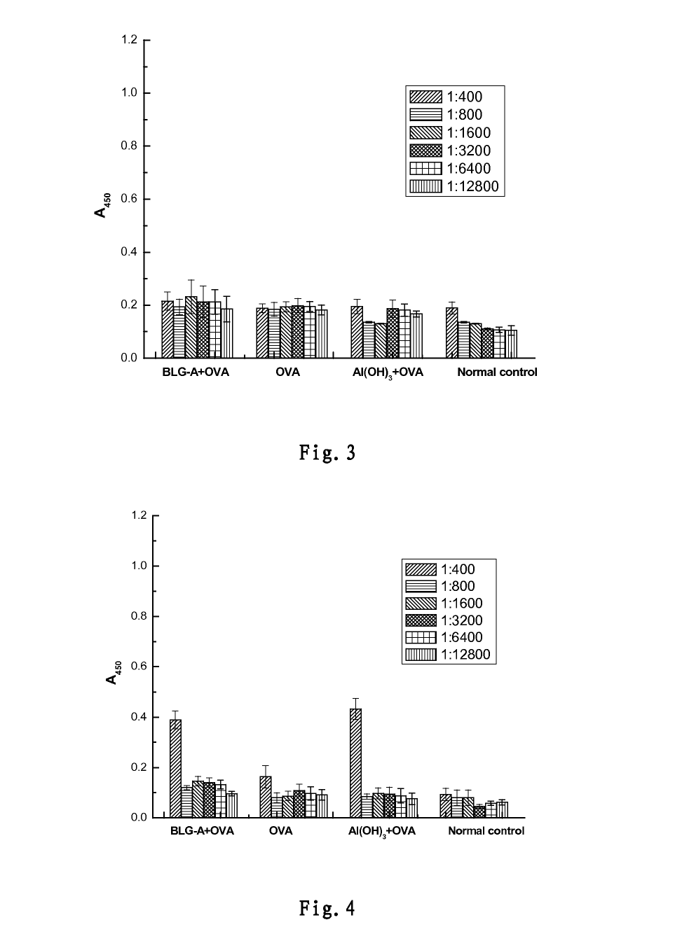

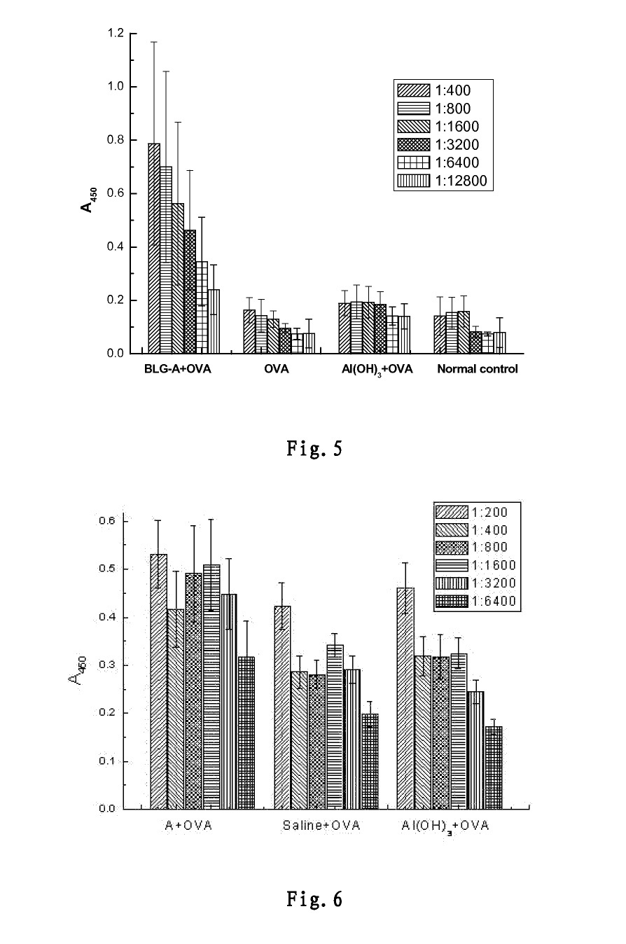

Experimental results showed that after the first and second immunization, the antibody titres of all measured groups were relatively low (FIG. 3 and FIG. 4). After the third immunization, BLG-A injection animal blood serum had a relatively higher antibody titre (FIG. 5), which indicated BLG-A could significantly promote antibody generation, the effect of adjuvant was superior to that of aluminum adjuvant (FIG. 3, FIG. 4 and FIG. 5 respectively showed anti-OVA antibody titres in mouse blood serum as measured by ELISA after the 1^{st}, 2^{nd} and 3^{rd} immunization, mean±SD; n=5).

### Example 6

### Measurement of Adjuvant Activity of Radix Isatidis Total Polysaccharide Sample 2

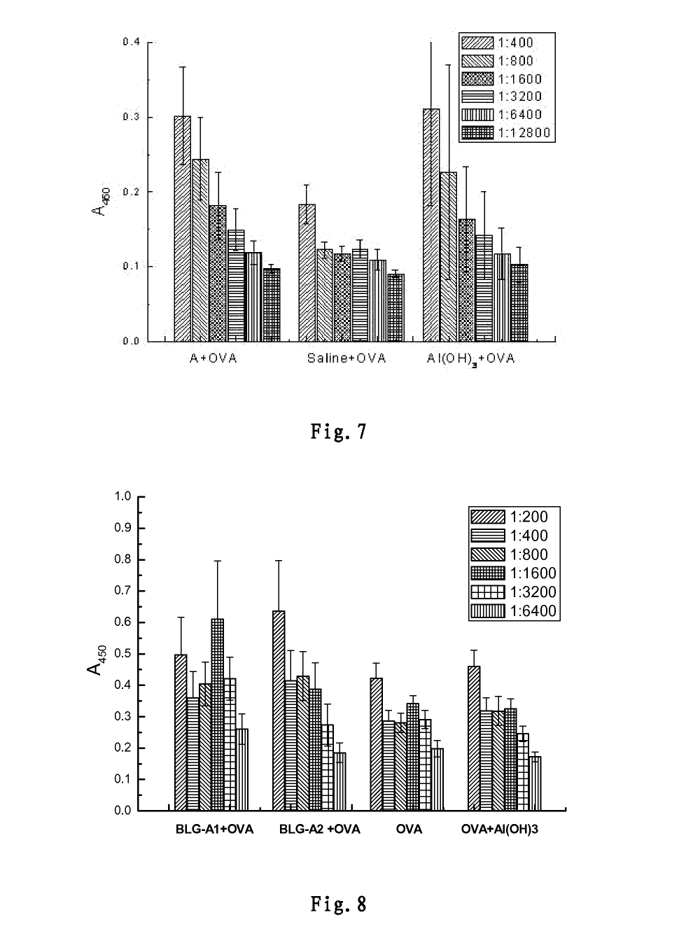

Specific steps were identical to those of Example 5, except that the used sample was Radix isatidis total polysaccharide sample 2 as prepared in Example 2. The results were shown in FIG. 6, FIG. 7.

The results showed that Radix isatidis total polysaccharide sample 2 could effectively promote antibody generation.

### Example 7

### Determination of Antibody Titres of Radix Isatidis Neutral Polysaccharide Fraction and Acidic Polysaccharide Fraction (1)

1. Experimental Objective:

The Radix isatidis neutral polysaccharide fraction BLG-A1 and acidic polysaccharide BLG-A2 as prepared in Example 3 were used as adjuvants, ovalbumin (OVA) was used as antigen, both of them in combination were intramuscularly injected to mice, and the generated antibody titre was determined.

2. Experimental Methods

Specific experimental steps referred to Example 5.

Drug concentration: BLG-A1: 10 mg/ml; BLG-A2: 10 mg/ml; OVA: 1.2 mg/ml; aluminum adjuvant: 2 mg/ml; control solvent: physiological saline.

Administration dose: OVA-60 μg/50 μl/mouse; aluminum adjuvant-100 μg/50 μl/mouse;

BLG-A1: 0.5 mg/50 μl/mouse; BLG-A2: 0.5 mg/50 μl/mouse;

Grouping: (1) P group: PBS+OVA; (2) aluminum adjuvant group: aluminum adjuvant+OVA; (3) BLG-A1 group: BLG-A1+OVA; (4) BLG-A2 group: BLG-A2+OVA;

Mixing in equal-volume was performed before injection, 100 μl/mouse, intramuscular injection was performed at right hindlimb.

3. Experimental Results

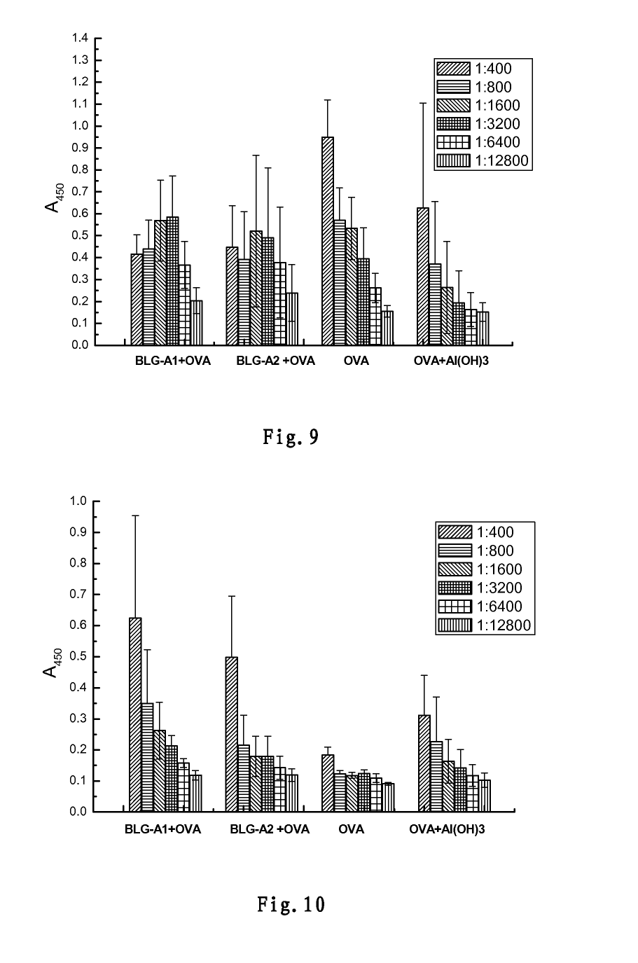

The Radix isatidis total polysaccharide BLG-A was separated by DEAE-cellulose column chromatography to obtain neutral polysaccharide fraction BLG-A1 and acidic polysaccharide fraction BLG-A2. Adjuvant activity and function were further determined according to immunization schedule, assay was performed after the 1^{st} and 2^{nd} immunization, A1 and A2 fractions had relatively high immune adjuvant activity (FIG. 8 and FIG. 9). After the 3^{rd} immunization, the antibody titre did not increase significantly (FIG. 8, FIG. 9 and FIG. 10 respectively were blood serum anti-OVA antibody titre as assayed by ELISA in mice after the 1^{st}, 2^{nd} and 3^{rd} immunization, mean±SD; n=5).

### Example 8

### Determination of Antibody Titres of Radix Isatidis Neutral Polysaccharide Fraction and Acidic Polysaccharide Fraction (2)

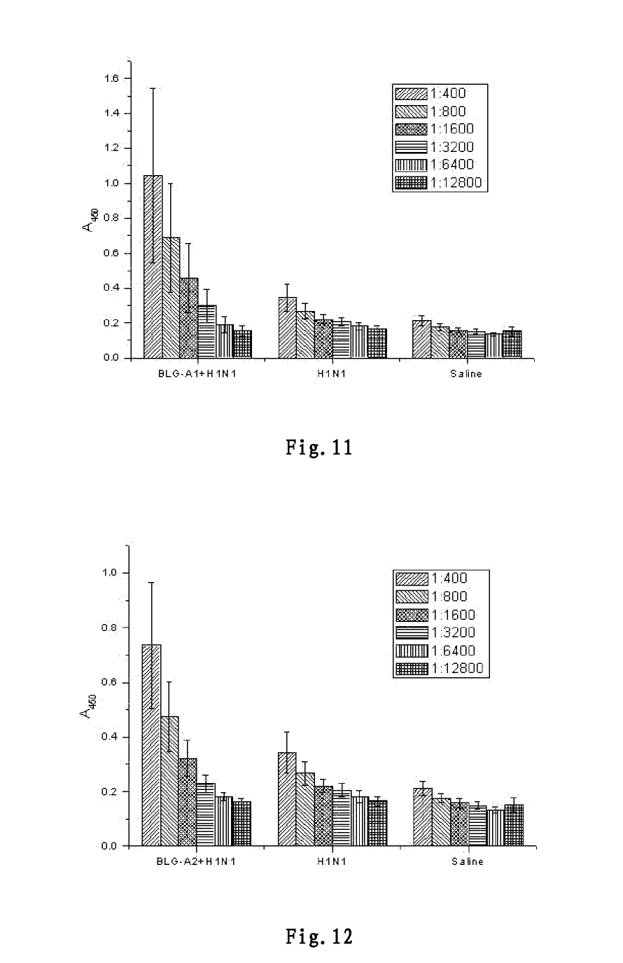

The Radix isatidis neutral polysaccharide fraction BLG-A1 and acidic polysaccharide fractionBLG-A2 as prepared in Example 3 were respectively used as adjuvants, mixed with H1N1 influenza vaccine (H1N1 influenza virus lysate, 30 μg/ml), and used for immunizing mice. And antibody titre was also determined by ELISA method after 2 weeks. The experiment was performed in physiological saline group, H1N1 vaccine group and H1N1+BLG-A1 group, and the results showed when H1N1 virus lysate was used as immune antigen, BLG-A1 and BLG-A2 as adjuvant, primary immunization could generate antibody in relatively high titre (FIG. 11, FIG. 12).

### Example 9

### Determination of Antibody Titres of Radix Isatidis Neutral Polysaccharide Fraction and Acidic Polysaccharide Fraction (3)

The used sample: the Radix isatidis total polysaccharide sample 1 of Example 1 was processed according to the method of Example 3 to respectively obtain Radix isatidis neutral polysaccharide fraction and acidic polysaccharide fraction.

Experimental steps referred to Example 8.

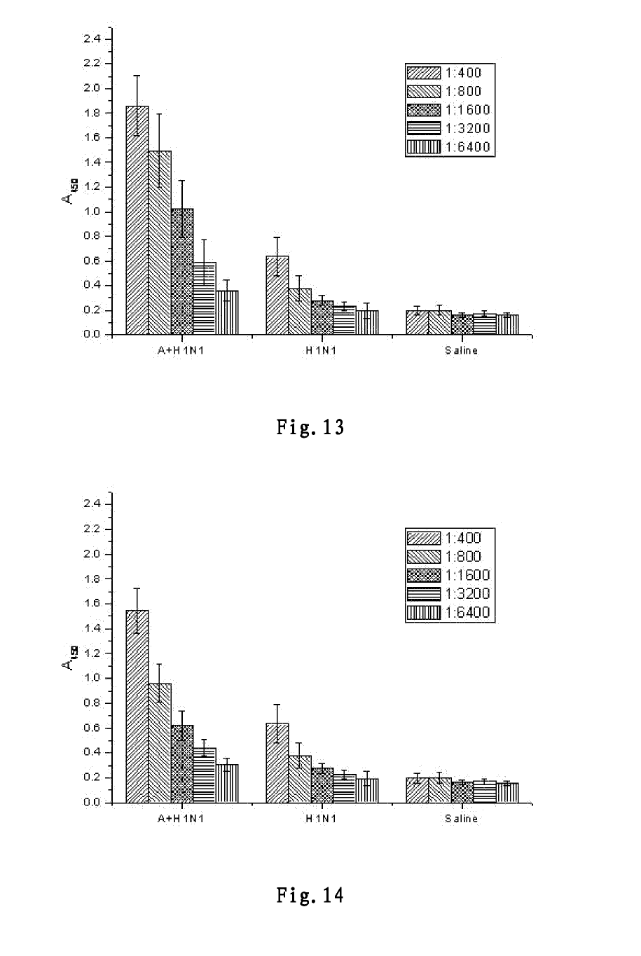

The results were shown in FIG. 13, and FIG. 14.

The results showed that the Radix isatidis neutral polysaccharide fraction and acidic polysaccharide fraction as prepared from the Radix isatidis total polysaccharide sample 1 could effectively promote antibody generation.

Although the embodiments of the present invention are described in details, those skilled in the art would understand that these details could be modified and changed according to the teachings of the disclosures, and these modifications and changes are all in the protection scope of the present invention. The scope of the present invention is given by the attached claims and any equivalents thereof.

What is claimed is:

1. A Radix isatidis polysaccharide fraction having the following characteristics:
(1) which comprises glucose, galactose, mannose, rhamnose, arabinose and xylose, and has molar ratio of Rha:Ara:Xyl:Man:Glc:Gal=1.00:2.35:2.38:9.27:27.47:13.03;
(2) expressed in glucose, saccharide content is 98.13%;
(3) molecular weight is 2000-10000.

2. A Radix isatidis polysaccharide fraction having the following characteristics:
(1) which comprises arabinose, glucose, galactose, rhamnose and mannose, and has molar ratio of Rha:Ara:Man:Glc:Gal=1.00:40.06:0.61:22.24:18.04;
(2) expressed in glucose, saccharide content is 92.11%;
(3) expressed in galactose-uronic acid, glycuronic acid content is 6.41%;
(4) molecular weight is 3000-70000.

3. A Radix isatidis total polysaccharide, which comprises:
(1) the Radix isatidis polysaccharide fraction of claim 1; and
(2) the Radix isatidis polysaccharide fraction of claim 2.

4. The Radix isatidis total polysaccharide according to claim 3, characterized in that:
(1) expressed in glucose, saccharide content is 58.93%;
(2) expressed in galactose-uronic acid, glycuronic acid content is 13.36%.

5. The Radix isatidis total polysaccharide according to claim 4, which has the characteristics as shown in FIG. 1 or FIG. 2.

6. The Radix isatidis total polysaccharide according to any one of claims 3-5, which is prepared by the following steps:
1) extracting Radix isatidis with water at 50° C.-55° C., to obtain a aqueous extract;
2) subjecting the aqueous extract of step 1) to ethanol precipitation, dialyzing and lyophilizing supernatant, to obtain the Radix isatidis total polysaccharide.

7. The Radix isatidis total polysaccharide according to claim 6, characterized in one or more of the following items (1)-(12):
(1) in step 1), the residue obtained after extraction is subjected to extraction under same conditions once or more times, and aqueous extracts are combined;
(2) in step 1), the used water is distilled water or deionized water;
(3) in step 1), the water is in an amount of 5-15 times of Radix isatidis (L/Kg);
(4) in step 1), the used Radix isatidis is pulverized Radix isatidis;
(5) in step 1), the used Radix isatidis is Radix isatidis residue extracted with an organic solvent (such as, petroleum ether, ethyl acetate, chloroform, ethyl ether, n-hexane, cyclohexane, n-butanol, ethanol or methanol);
(6) in step 1), stirring is performed during extraction period;
(7) in step 1), the obtained aqueous extract is subjected to vacuum concentration to obtain a concentrated aqueous extract;
(8) in step 2), conditions for ethanol precipitation are: after ethanol precipitation, final ethanol concentration is 60-80%; preferably, ethanol precipitation time is greater than 12 h;
(9) in step 2), the precipitate obtained by centrifugation after ethanol precipitation is further subjected to ethanol precipitation once or more times, and the supernatants are combined;
(10) in step 2), dialysis bag used for dialysis has molecular cut off of greater than 1000;
(11) in step 2), the dialysis is performed once or more times;
(12) in step 2), before lyophilization, the obtained dialysate is concentrated at 50° C.-55° C.

8. The Radix isatidis polysaccharide fraction according to claim 1, which is obtained by the following steps:
subjecting the Radix isatidis total polysaccharide of claim 6 or 7 to DEAE-cellulose column chromatography to obtain a water elution portion.

9. The Radix isatidis polysaccharide fraction according to claim 2, which is prepared by the following steps:
subjecting the Radix isatidis total polysaccharide of claim 6 or 7 to DEAE-cellulose column chromatography to obtain a 0.25 NaHCO₃ elution portion.

10. A pharmaceutical composition, which comprises the Radix isatidis polysaccharide fraction or Radix isatidis polysaccharide of any one of claims 1 to 9; optionally, further comprises a pharmaceutically acceptable excipient.

11. A vaccine adjuvant, which comprises the Radix isatidis polysaccharide fraction and/or Radix isatidis total polysaccharide of any one of claims 1 to 9; specifically, the vaccine adjuvant is an adjuvant for attenuated vaccines, protein vaccines, DNA vaccines or polypeptide vaccines.

12. A vaccine preparation or vaccine composition, which comprises the Radix isatidis polysaccharide fraction and/or Radix isatidis total polysaccharide of any one of claims 1 to 9; specifically, the vaccine preparation or vaccine composition is an attenuated vaccine, protein vaccine, DNA vaccine or polypeptide vaccine; more specifically, is H1N1 influenza vaccine.

13. Use of the Radix isatidis polysaccharide fraction or Radix isatidis total polysaccharide of any one of claims 1 to 9 as a vaccine adjuvant; or use in manufacture of a vaccine preparation, vaccine composition, or antibody.

14. The use according to claim 13, wherein the vaccine preparation is an attenuated vaccine, protein vaccine, DNA vaccine or polypeptide vaccine; the vaccine adjuvant is an adjuvant for attenuated vaccines, protein vaccines, DNA vaccines or polypeptide vaccines.

15. A method for preparing an antibody, comprising a step of using an effective amount of the Radix isatidis polysaccharide fraction and/or Radix isatidis total polysaccharide of any one of claims 1 to 9; specifically, the antibody is a monoclonal antibody or polyclonal antibody.

16. A method for immunization or vaccination, comprising administering a mammal with an effective amount of the vaccine preparation or vaccine composition of claim 12.
